SEQUENCE LISTING (32 sequences)

SEQ ID NO: 1 - DNA, 24 bp, Artificial Sequence
  Description of Artificial Sequence Synthetic primer
aggaccgcggttctattttgttgg

SEQ ID NO: 2 - DNA, 24 bp, Artificial Sequence
  Description of Artificial Sequence Synthetic primer
atgctttcgctctggtccgtcttg

SEQ ID NO: 3 - DNA, 21 bp, Artificial Sequence
  Description of Artificial Sequence Synthetic primer
ccaccacacctgaaagcagaa

SEQ ID NO: 4 - DNA, 20 bp, Artificial Sequence
  Description of Artificial Sequence Synthetic primer
aggtgaaaggcagggactca

SEQ ID NO: 5 - DNA, 21 bp, Artificial Sequence
  Description of Artificial Sequence Synthetic primer
tggccgggtctgctgagtccg

SEQ ID NO: 6 - DNA, 21 bp, Artificial Sequence
  Description of Artificial Sequence Synthetic primer
gtccatgggaagatgttctgg

SEQ ID NO: 7 - DNA, 21 bp, Artificial Sequence
  Description of Artificial Sequence Synthetic primer
ctgagtccgaatcaggtgcag

SEQ ID NO: 8 - DNA, 21 bp, Artificial Sequence
  Description of Artificial Sequence Synthetic primer
gtccatgggaagatgttctgg

SEQ ID NO: 9 - DNA, 20 bp, Artificial Sequence
  Description of Artificial Sequence Synthetic primer
cagcactcagactatgtgca

SEQ ID NO: 10 - DNA, 21 bp, Artificial Sequence
  Description of Artificial Sequence Synthetic primer
gtccatgggaagatgttctgg

SEQ ID NO: 11 - DNA, 20 bp, Artificial Sequence
  Description of Artificial Sequence Synthetic primer
gggttctgtcttccactcca

SEQ ID NO: 12 - DNA, 21 bp, Artificial Sequence
  Description of Artificial Sequence Synthetic primer
aagcagcagagtcaggctttc

SEQ ID NO: 13 - DNA, 20 bp, Artificial Sequence
  Description of Artificial Sequence Synthetic primer
tctggggcaaatttgtgctg

SEQ ID NO: 14 - DNA, 20 bp, Artificial Sequence
  Description of Artificial Sequence Synthetic primer
agccacactttcatgttggc

SEQ ID NO: 15 - DNA, 20 bp, Artificial Sequence
  Description of Artificial Sequence Synthetic primer
tgctgcaaagtgaacaccag

SEQ ID NO: 16 - DNA, 20 bp, Artificial Sequence
  Description of Artificial Sequence Synthetic primer
atgcggtaacaggttttgcg

SEQ ID NO: 17 - DNA, 20 bp, Artificial Sequence
  Description of Artificial Sequence Synthetic primer
tcaatgctggcttcaaggag

SEQ ID NO: 18 - DNA, 20 bp, Artificial Sequence
  Description of Artificial Sequence Synthetic primer
agcgccttgttttgctgttc

SEQ ID NO: 19 - DNA, 19 bp, Artificial Sequence
  Description of Artificial Sequence Synthetic primer
gcggctgagaaatcacgtt

SEQ ID NO: 20 - DNA, 20 bp, Artificial Sequence
  Description of Artificial Sequence Synthetic primer
tcagtggttcaccgcttctt

SEQ ID NO: 21 - DNA, 24 bp, Artificial Sequence
  Description of Artificial Sequence Synthetic primer
caccagtgtgaattacagcaaatc

SEQ ID NO: 22 - DNA, 22 bp, Artificial Sequence
  Description of Artificial Sequence Synthetic primer
acaggagaatctcccagagttc

SEQ ID NO: 23 - DNA, 28 bp, Artificial Sequence
  Description of Artificial Sequence Synthetic primer
ggtttggagatggttatacaatagttgt

SEQ ID NO: 24 - DNA, 17 bp, Artificial Sequence
  Description of Artificial Sequence Synthetic primer
cccggaaacgcaagtcc

SEQ ID NO: 25 - DNA, 20 bp, Artificial Sequence
  Description of Artificial Sequence Synthetic primer
gacaccacctgtagggagga

SEQ ID NO: 26 - DNA, 20 bp, Artificial Sequence
  Description of Artificial Sequence Synthetic primer
agtaccaagcaggcaggaga

SEQ ID NO: 27 - DNA, 20 bp, Artificial Sequence
  Description of Artificial Sequence Synthetic primer
accttctacaatgagctgcg

SEQ ID NO: 28 - DNA, 20 bp, Artificial Sequence
  Description of Artificial Sequence Synthetic primer
ctggatggctacgtacatgg

SEQ ID NO: 29 - DNA, 20 bp, Artificial Sequence
  Description of Artificial Sequence Synthetic primer
agagcttcgtccacaaaagc

SEQ ID NO: 30 - DNA, 20 bp, Artificial Sequence
  Description of Artificial Sequence Synthetic primer
agcacgttgtaatggaagcc

SEQ ID NO: 31 - DNA, 20 bp, Artificial Sequence
  Description of Artificial Sequence Synthetic primer
ttgtggactttgccaagcag

SEQ ID NO: 32 - DNA, 20 bp, Artificial Sequence
  Description of Artificial Sequence Synthetic primer
tgcattctgtctcgtggttg

(19) United States
(12) Patent Application Publication
(10) Pub. No.: US 2020/0038410 A1
(43) Pub. Date: Feb. 6, 2020

(54) METHODS FOR TREATING NEUROPATHY

(71) Applicant: Loyola University Chicago, Maywood, IL (US)

(72) Inventor: Virginie Mansuy-Aubert, Chicago, IL (US)

(21) Appl. No.: 16/522,774

(22) Filed: Jul. 26, 2019

Related U.S. Application Data
(60) Provisional application No. 62/703,720, filed on Jul. 26, 2018.

(51) Int. Cl. A61K 31/56; A61P 3/00

(52) U.S. Cl. CPC A61K 31/56; A61K 45/06; A61P 3/00

(57) ABSTRACT

Methods of treating, controlling, and delaying the onset and progression of neuropathy, including neuropathic pain associated with metabolic syndrome, including but not limited to obesity associated therewith. The methods include administering a liver X receptor agonist to the subject.

### CROSS REFERENCE TO RELATED APPLICATIONS

This application claims the benefit of U.S. Provisional Application No. 62/703,720, filed Jul. 26, 2018, the contents of which are incorporated herein by reference.

### BACKGROUND OF THE INVENTION

The present invention generally relates to neuropathy and methods of treating or controlling neuropathy. The invention particularly relates to methods of studying, treating, controlling, and delaying the onset and progression of neuropathy, including neuropathic pain associated with metabolic syndrome, including but not limited to obesity associated therewith.

Obesity, which has reached epidemic proportions in the United States and is increasing worldwide, is associated with insulin resistance, type 2 diabetes, dyslipidemias, cardiovascular pathologies, and neurodegenerative disorders. This constellation of symptoms, collectively termed metabolic syndrome, continues to rise, particularly in countries adopting westernized diets. More than half of the patients with diabetes, alone or in combination with other components of metabolic syndrome, often develop some form of type 2 diabetic peripheral neuropathy. The pathophysiology of diabetic neuropathy is complex and still under debate. There is a recent body of evidence linking painful neuropathy to obesity, independent of diabetes, and highlighting the importance of lipid metabolism in the onset of neuropathy. Because of this complexity, there are still no known pharmacological treatments that target peripheral neuropathy.

In view of the above, it can be appreciated that there is an ongoing desire for improved methods relating to the treatment of subjects for neuropathy, including but not limited to methods of studying, treating, controlling, and delaying the onset and progression of neuropathy associated with obesity.

### BRIEF DESCRIPTION OF THE INVENTION

The present invention provides methods of studying, treating, controlling, and delaying the onset and progression of neuropathy, including neuropathic pain associated with metabolic syndrome, including but not limited to obesity associated therewith.

According to one aspect of the invention, a method is provided for treating neuropathy in a subject that includes administering a liver X receptor agonist to the subject.

According to one aspect of the invention, a method is provided for treating neuropathy in a subject that includes administering a liver X receptor agonist to the subject in an amount sufficient to control endoplasmic reticulum stress due to the accumulation of unfolded proteins.

According to yet another aspect of the invention, methods as described above are used to control and optionally delay the onset and progression of neuropathy, and in particular neuropathy associated with metabolic syndrome.

Technical effects of the methods described above preferably include the capability of studying and treating neuropathy relating to metabolic syndrome, including but not limited to obesity associated therewith. In particular, it is believed that, in regard to pain due to conditions including metabolic syndrome, obesity, aging, and skin condition (e.g., inflammation of the skin lead to pain that uses the same fibers that allodynia), all LXR agonists (particularly but not limited to GW3965, T0901317, desmosterol, N,N-dimethyl-3β-hydroxycholenamide (DMHCA) and methylpiperidinyl-3β-hydroxycholenamide (MePipHCA), cholesterol, and hydroxycholesterol), when administered intraperitoneally, intravenous, orally, or topically can improve neuropathy and pain associated therewith.

Other aspects and advantages of this invention will be further appreciated from the following detailed description.

### BRIEF DESCRIPTION OF THE DRAWINGS

FIGS. 1A through 1G include charts and graphs representing data indicating that a liver X receptor (LXR, LXRα and LXRβ) agonist (GW3965) regulates dorsal root ganglia (DRG) gene expression and protects from palmitate-induced endoplasmic reticulum (ER) stress in the DRG. FIG. 1A is a chart representing distribution of nuclear receptor mRNA in the whole DRG. Normalized mRNA expression levels were defined as Absent if the Ct value was greater than 40, Low if the level was greater than 0.025 arbitrary units, Moderate if the level was between 0.025 and 0.25, and High if the level was greater than 0.25 arbitrary units. FIG. 1B represents data indicating that the LXR agonist increased gene expression of LXR targets in organotypic cultures of DRG. FIGS. 1D and 1F represent data of protein levels of CHOP, an ER stress marker, in DRG of mice fed western diet (WD) compared to normal chow (NC) (FIG. 1D, n=10 DRG/group), and in ex vivo organotypic whole DRG cultures treated with palmitate and LXR agonist (FIG. 1F, n=individual experiments in triplicate). FIGS. 1C, 1E, and 1G represent data of mRNA levels of ER stress markers using 18S to normalize, in DRG of WD and NC fed mice (FIG. 1C, n=4 mice/group), in organotypic whole DRG cultures (FIG. 1E), and in primary neuronal culture of DRG neurons treated with LXR agonist and palmitate (FIG. 1G, n=5 individual experiments). (All values are Mean±S.E.M, with vehicle group defined as 100%; *p<0.05 with vehicle; **p<0.05 with vehicle+palmitate).

FIGS. 2A through 2D include charts representing data indicating that an LXR agonist (GW3965) delayed the progression of western diet-induced allodynia and protects the DRG from ER stress. FIG. 2A shows data from von Frey tests to assess sensitivity of mice on either diet treated with LXR agonist to innocuous stimuli (Week1=1 week after agonist admission, 9 weeks on WD, 13 weeks of age). End point levels of serum triglycerides (FIG. 2B), cholesterol (FIG. 2C), in mice fed NC or WD treated with agonist. FIG. 2D represents mRNA levels of ER stress markers normalized to 18S in DRG of NC or WD-fed mice treated with LXR agonist. (n=4 mice/group; all values are Mean±S.E.M; for mRNA relative levels were plotted with NC-vehicle group defined as 100%; *p<0.05 with NC-Veh; **p<0.05 with WD-Veh).

FIGS. 3A through 3F include charts and images representing that an LXR agonist decreased lipid-induced ER stress in DRG neurons expressing Nav1.8. FIG. 3A shows data of von Frey tests to assess sensitivity of LXRab and LXRabnav mice on either diet to innocuous stimuli (n=5/group), *p<0.05 compared to LXRab NC, **p<0.05 compared to LXRabnav, # p<0.05 compared to LXRab WD. FIG. 3B represents the generation of tissue specific RiboTag mouse. Sensory neuron specific (Nav1.8) Cre mice were utilized to generate Ribotag-Nav1.8-Cre mice. FIG. 3C represents data from western blots on whole DRG of RiboTag-Nav1.8-Cre mice after immunoprecipitation for HA. FIG. 3D represents data from immunohistochemistry on DRG slices for HA in sensory neurons (in green—HA, blue—DAPI/nuclei). FIG. 3E represents actin normalized mRNA levels of positive (Nav1.8) and negative (GFAP, PV) markers of Nav1.8 expressing neurons in whole DRG (WT), input, and IP samples. FIG. 3F represents actin normalized mRNA levels of ER stress markers, in sensory neurons treated with LXR agonist and palmitate. Sensory neuron specific mRNA was isolated by immunoprecipitation from whole DRG organotypic cultures treated with agonist and palmitate (n=3 individual experiments). (Values are Mean±S.E.M, with vehicle group defined as 100%; *p<0.05 with vehicle; **p<0.05 with vehicle+palmitate).

FIGS. 4A through 4K include charts and images representing western diet induces obesity, lipid accumulation, and allodynia. FIG. 4A represents body weight of mice on normal diet (NC) and western diet (WD) over twelve weeks. FIG. 4B represents intraperitoneal glucose tolerance test (GTT) of NC, and, WD-fed mice. FIG. 4C represents intraperitoneal insulin tolerance test of NC, and, WD-fed mice. FIGS. 4D through 4G represent levels of serum triglycerides (FIG. 4D), cholesterol (FIG. 4E), leptin (FIG. 4F), and insulin (FIG. 4G) in WD and NC-fed mice at end of twelve weeks. FIG. 4H represents data from von Frey tests to assess allodynia, relative threshold values represented with mean 50% threshold of NC mice as 1. FIG. 4I shows mice fed on either normal diet (NC) or western diet (WD) for twelve weeks. FIG. 4J shows livers of NC and WD mice after twelve week of diet. FIG. 4K shows H and E staining on liver sections of NC and WD mice. (All values are Mean±S.E.M; n=8/group; *p<0.05 with respect to NC controls).

FIGS. 5A through 5C include charts representing (FIG. 5A) body weights of LXRab (control) and sensory neuron specific LXRab knockout (LXRabnav) after sixteen weeks on normal (NC) and western diet (WD); *p<0.05 compared to LXRab NC, **p<0.05 compared to LXRabnav NC, # p<0.05 compared to LXRab WD mice, (FIG. 5B) mRNA expression of LXRa and LXRb in DRG of LXRab and LXRabnav mice show marked reduction of expression in knockout mice, *p<0.05 compared to LXRab mice, and (FIG. 5C) bioanalyzer trace of mRNA from DRG samples of whole ganglia, RiboTag-Nav1.8-Cre (input, IP-negative control, and IP-HA).

FIG. 6 is a bar chart evidencing that treatments with a mixed solution of butyrate and an LXR agonist (GW3965) trigger an enhanced regeneration mechanism that improves neuropathy.

### DETAILED DESCRIPTION OF THE INVENTION

Disclosed herein are methods of studying, treating, and controlling neuropathy associated with metabolic syndrome, including but not limited to obesity associated therewith. The methods include administering one or more liver X receptors (LXR) ligands (hereinafter referred to as agonists) to a subject, as a nonlimiting example, to delay or treat a western diet-induced allodynia. The LXR agonist may be administered by various methods known in the art such as but not limited to injection or in application of a topical compound (for example, microparticles in a lotion) and may be administered with various carriers and other active or inactive compounds, for example, in a mixture further including any form of butyrate.

Understanding the early cell-specific mechanisms underlying a metabolism-induced pathology is critical for developing therapeutic treatments. One such mechanism involves the endoplasmic reticulum (ER), the organelle responsible for protein folding and trafficking. When the ER becomes stressed due to the accumulation of unfolded proteins, the unfolded protein response (UPR) is activated. The UPR regulates the ER by synthesis of lipids and protein components of the ER to meet varying demands on protein folding in response to pathophysiological conditions. The ER, in addition to housing proteins involved in lipid metabolism, is also the major site for the synthesis of sterols and phospholipids and regulates membrane lipid homeostasis. It has been previously reported that obesity induces ER stress in various tissues including neurons, which in turn leads to insulin resistance and type 2 diabetes. Additionally, evidence has suggested ER stress in neurons of the peripheral nervous system (PNS) may be a potential mechanism in the onset and progression of allodynia. It was therefore theorized that modulating ER stress in PNS could prevent or reduce lipotoxicity and attenuate the progression of neuropathy induced by metabolic diseases.

As used herein, LXR (or LXRs) refers to liver X receptors, including its two identified isoforms referred to as LXRα and LXRβ. LXRs are lipid activated transcription factors, and play a crucial role in regulation of cholesterol and fatty acid homeostasis. It is believed that the role of these receptors in central and peripheral nervous system has not been previously clarified using tissue-specific approaches. Investigations leading to aspects of the present invention (described below), indicate that LXR agonist treatment delays obesity-induced allodynia.

Nonlimiting embodiments of the invention will now be described in reference to experimental investigations leading up to the invention.

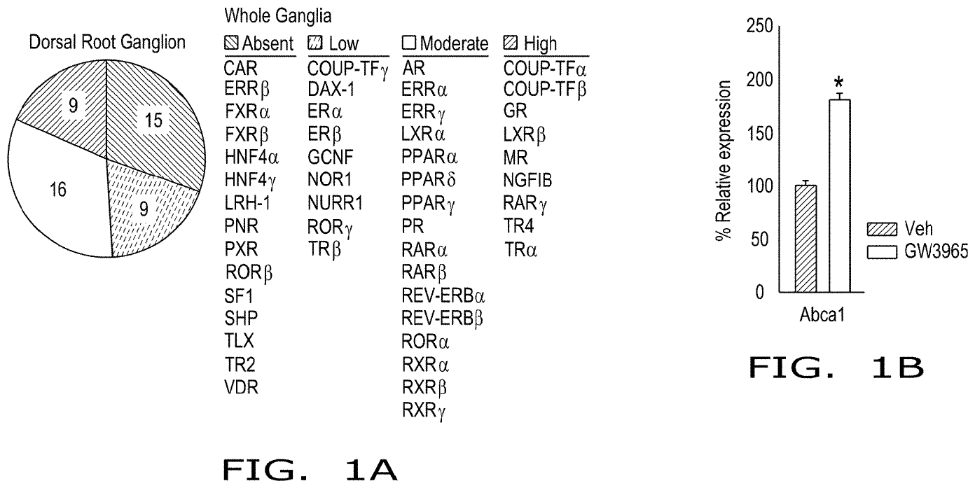

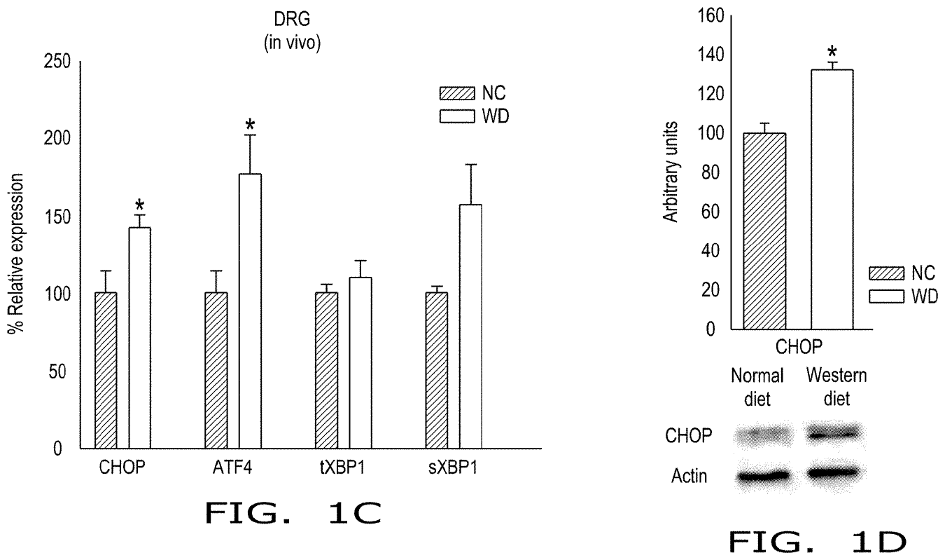

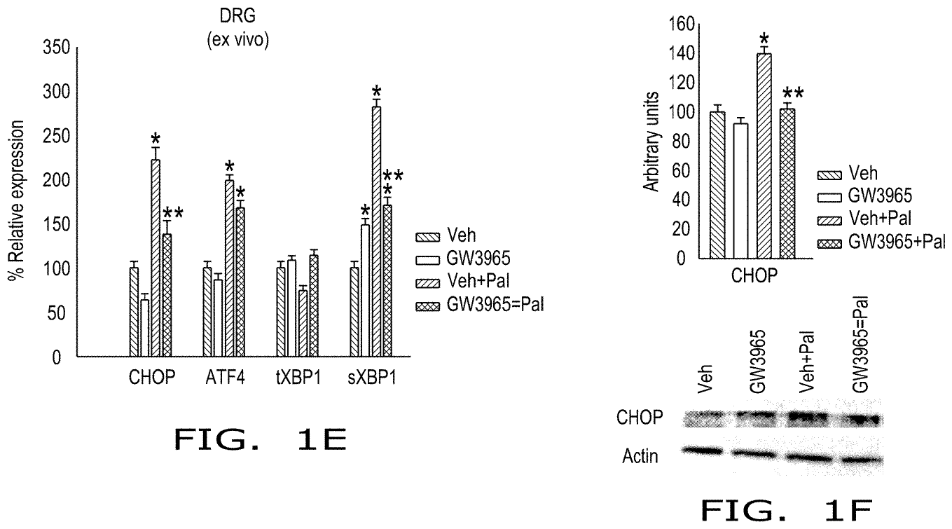

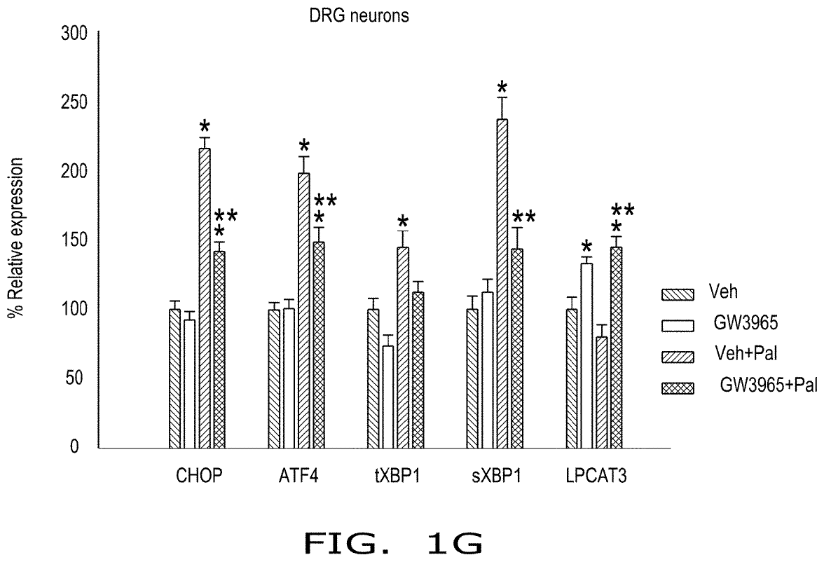

Nuclear receptors (NRs) are ligand-activated transcription factors that bind to lipophilic hormones and dietary-derived lipids to regulate essential metabolic, inflammatory, and oxidative pathways. A high-throughput real-time PCR screen was performed to investigate the expression pattern of the 49 murine NRs in the dorsal root ganglia (DRG) of wild-type (WT) mice. NRs were classified according to their expression levels and by physiological relevance (FIG. 1A). Analysis of the data showed that several NRs important in lipid homeostasis and inflammation were expressed at moderate to high levels in the DRG including LXRs.

LXRs (which include but are not limited to GW3965, T0901317, desmosterol, N,N-dimethyl-3β-hydroxycholenamide (DMHCA) and methylpiperidinyl-3β-hydroxycholenamide (MePipHCA), cholesterol, and hydroxycholesterol) are important regulators of cholesterol, fatty acid, and glucose homeostasis in many cell types. It was hypothesized that the LXR pathway may mediate certain aspects of lipid-remodeling leading to obesity-induced dysfunction of the DRG/sciatic nerve. A significant increase was observed of LXR canonical gene expression involved in cholesterol homeostasis, ATP-binding cassette transporter (ABCA1) in organotypic cultures of DRG stimulated with a liver X receptor full agonist (GW3965; FIG. 1B) confirming that LXRs are present and transcriptionally active in the DRG.

ER stress has been identified as a potential culprit underlying type 1 and type 2 diabetes. Increased expression of the ER stress marker CHOP was reported in metabolic tissues of diabetic mice, while targeted disruption of CHOP gene delayed the onset of diabetes. In addition, CHOP knock-out mice exhibit reduced oxidative stress and increased pancreatic cell survival in mouse models of diabetes. In the present investigations, an up-regulation in ER stress markers in the DRG of western diet (WD)-fed mice (TD88137; commercially available from Envigo under the product name Teklad; 42% kcal from fat, 34% sucrose by weight, and 0.2% cholesterol total) compared to control mice (Teklad LM-485) was identified (FIGS. 1C and 1D). Compared to NC-fed mice, WD-fed mice had higher levels of CHOP, ATF4, and sXBP1 expression in the DRG (FIG. 1C). Lipid overload, particularly saturated fatty acids such as palmitate, alter the composition and properties of the ER membrane, triggering the UPR. Palmitate stimulation of DRG organotypic cultures increased the levels of CHOP and ATF4, and also increased the formation of spliced X-box binding protein-1 (sXBP1) (FIG. 1E), which is involved in enhancing the folding capacity of the ER to minimize ER stress. Activation of LXRs has been shown to decrease lipotoxicity of saturated fatty acids and suppress the UPR signaling in the liver. Therefore it was hypothesize that LXRs could regulate lipid-induced ER stress in the DRG neurons.

GW3965 treatment decreased the mRNA levels of ER stress markers in palmitate treated organotypic DRG cultures compared to those treated with vehicle (FIG. 1E). Similar results were obtained when DRG primary neuronal cultures were treated with palmitate and GW3965 (FIG. 1G). These findings suggest that in DRG neurons, LXRs could modulate saturated fatty acids-induced ER modification. Previous research has shown that LXRs could be involved in the regulation of organelles/membrane phospholipid composition by regulating the expression of LPCAT3 (lysophospholipid acyltransferase). Interestingly, in the present investigations a significant increase of Ipcat3 mRNA in DRG neurons stimulated by LXR agonist was also observed (FIG. 1G) suggesting that Ipcat3 is also a target of LXR in DRG neurons. This data suggested that LXRs play a role in the regulation of the ER-dependent phospholipid composition of the nerve fibers crucial for channel distribution and nerve function.

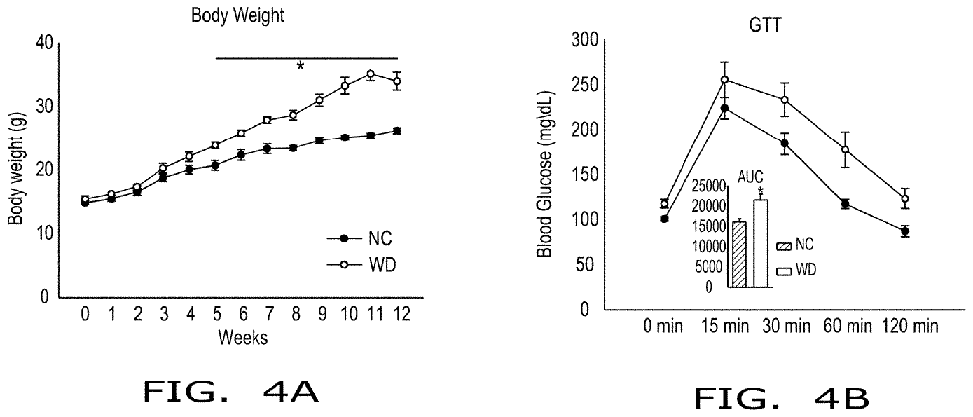

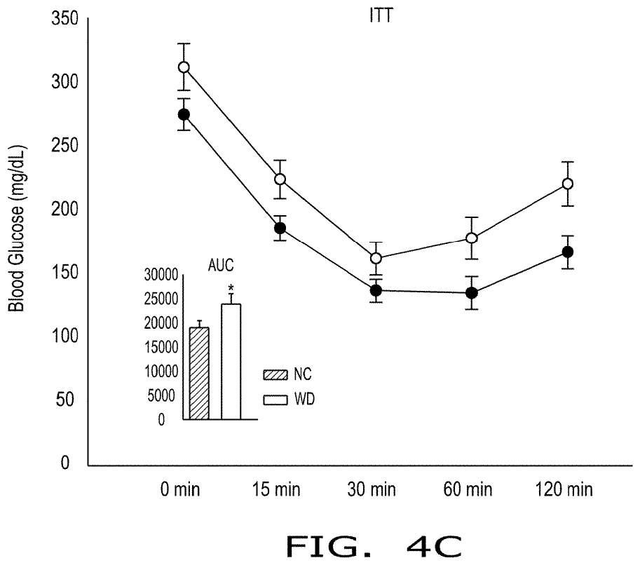

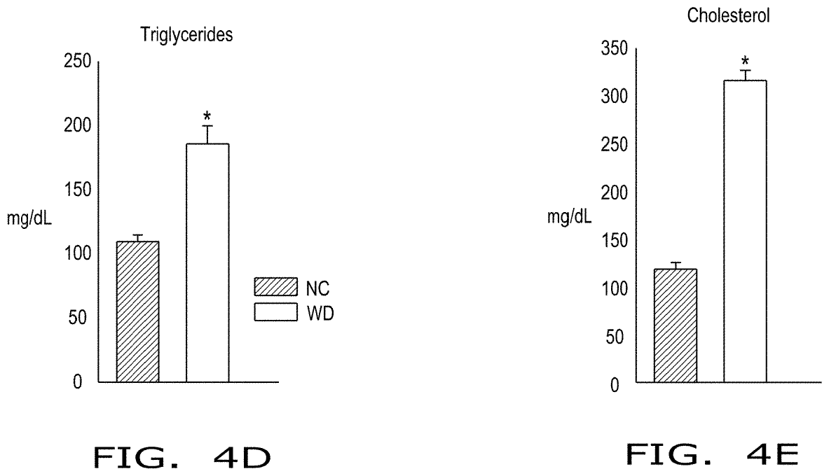

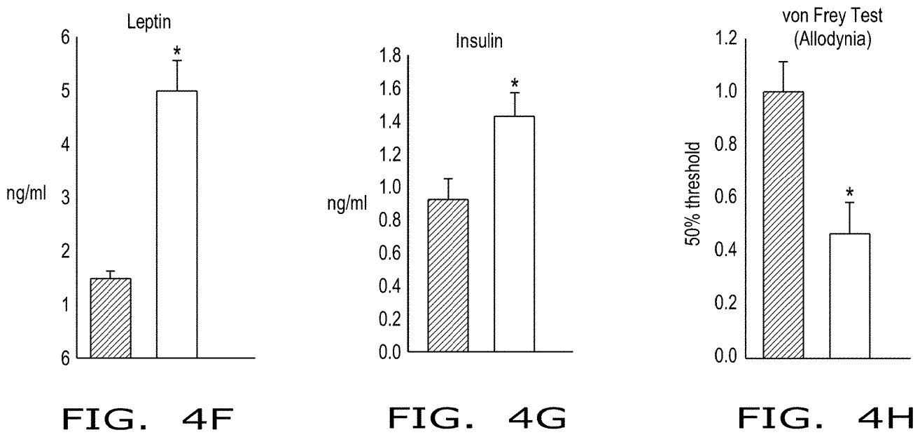

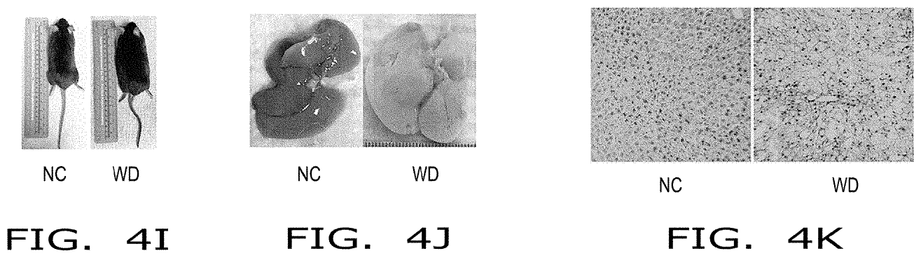

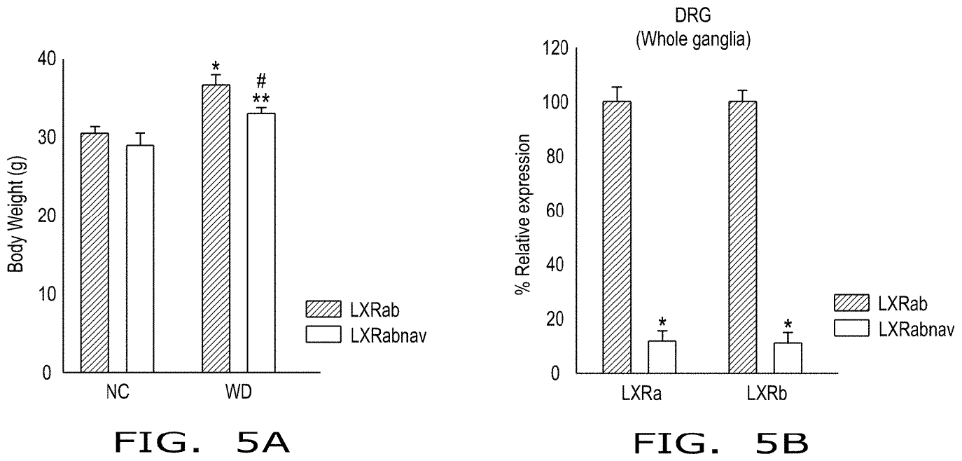

In order to determine the effect of an LXR agonist treatment on western diet allodynia, wild-type (WT) mice were maintained on a standard rodent diet (normal chow, NC) or western diet (high fat/high sucrose/high cholesterol, WD) for twelve weeks after weaning. The WD fed mice weighed significantly more after five weeks of WD (FIG. 4A). WD fed mice had significant higher levels of circulating insulin and leptin (FIGS. 4F and 4G), and showed impaired insulin sensitivity, during intraperitoneal glucose tolerance test or during insulin tolerance test (FIGS. 4B and 4C). Mice fed on WD also had higher levels of serum triglycerides and cholesterol, compared to mice fed NC (FIGS. 4D and 4E). Compared to NC control livers, livers of mice on WD also showed higher fat accumulation (FIGS. 4J and 4K).

The mechanical hypersensitivity observed early in peripheral neuropathy is believed to be associated with metabolic syndrome and independent of diabetes. This would suggest that the WD-fed model (obese and glucose intolerant) described herein represents an appropriate model to study the onset of peripheral neuropathy. The von Frey test was performed and measured phasic response frequency to calculate the 50% threshold in WD and NC-fed mice. Compared to NC mice, WD mice had a lower threshold (FIG. 4H) suggesting an increased sensitivity to innocuous stimuli. These findings suggest that WD mice represent a physiological model to study allodynia induced by obesity and metabolic syndrome.

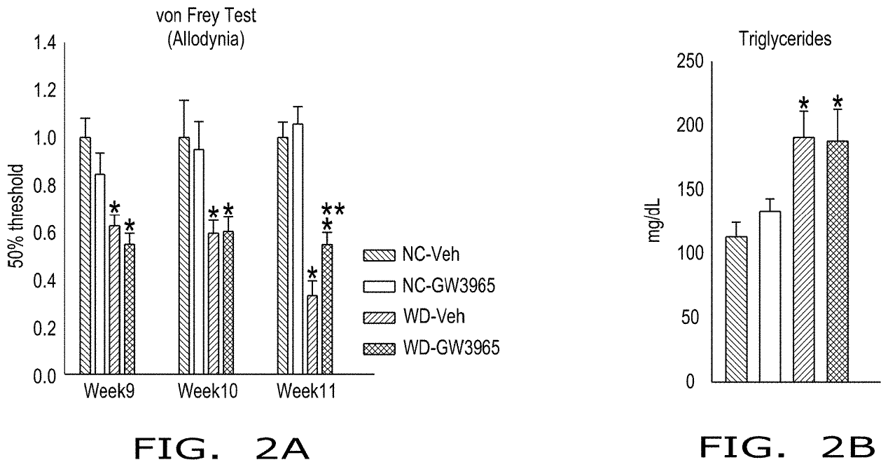

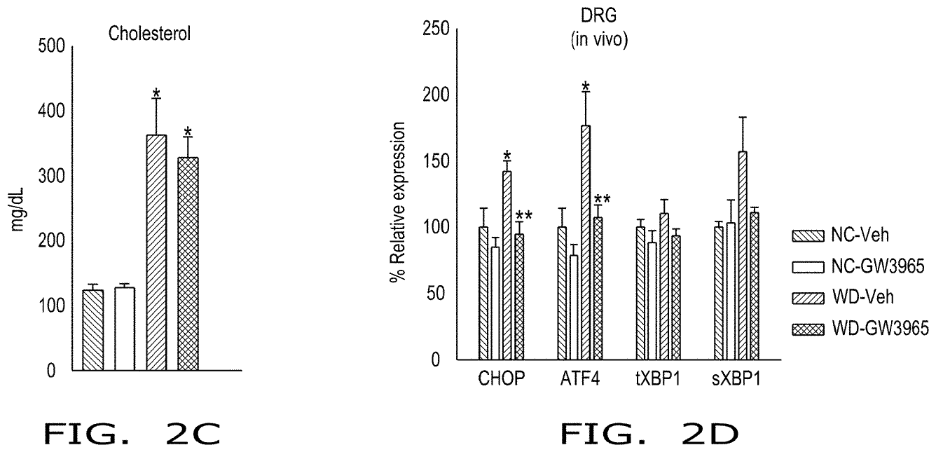

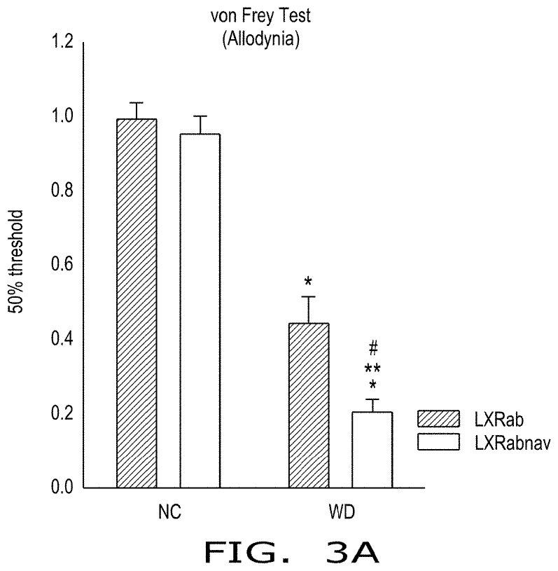

The above data linking LXR and ER stress in DRG led the inventors to assess whether activation of LXRs could change the WD-induced allodynia. WT mice were fed either NC or WD for a total of twelve weeks after weaning, while assessing the onset and progression of allodynia. WD-fed mice started exhibiting hypersensitivity within five weeks on WD reaching significant difference by week eight of WD (FIG. 2A). Weight-matched mice on either diet were treated for three weeks with either GW3965 (25 mg/kg body weight; twice a week by i.p.) or vehicle after eight weeks of WD diet (twelve weeks of age). As activation of LXRs elevated triglyceride levels in liver and plasma, the dosage of LXR agonist was adjusted to minimize the increase of triglyceride and cholesterol (FIGS. 2B and 2C). Sensitivity of mice to innocuous stimuli was evaluated over the duration of GW3965 treatment. Compared to WD mice injected with vehicle, WD mice with LXR agonist showed a delay in the progression of WD-induced allodynia (FIG. 2A). Then, the expression of UPR target genes in DRG of NC- or WD-fed mice treated with vehicle or GW3965 was compared. Activation of LXRs in WD-fed mice had decreased expression of ER stress markers (FIG. 2D). These findings suggested that LXR activation in the DRG can protect against WD-induced ER stress. This data also suggested that LXRs ameliorate WD-induced allodynia through the ER stress pathway.

The data suggested LXRs regulate diet-induced ER stress in the DRG. The DRG is a complex ganglion including different cell types including neurons, Schwann cells, immune cells, endothelial cells. To understand the molecular neurobiology underlying peripheral neuropathy, cell-specific approaches were used that, to the inventors knowledge, have never previously been reported in metabolic disease-induced neuropathy studies.

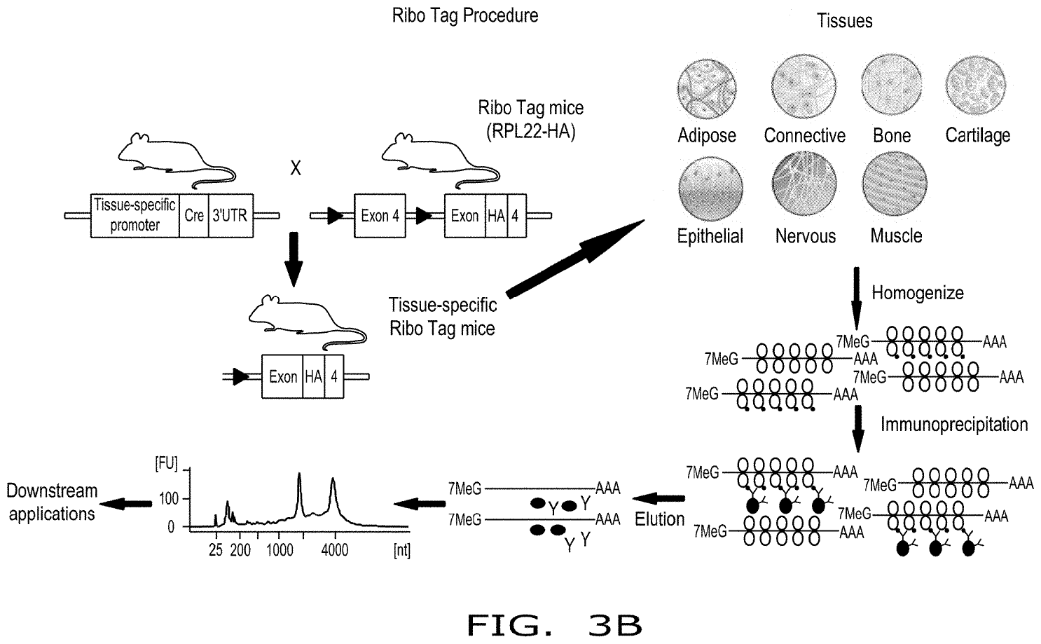

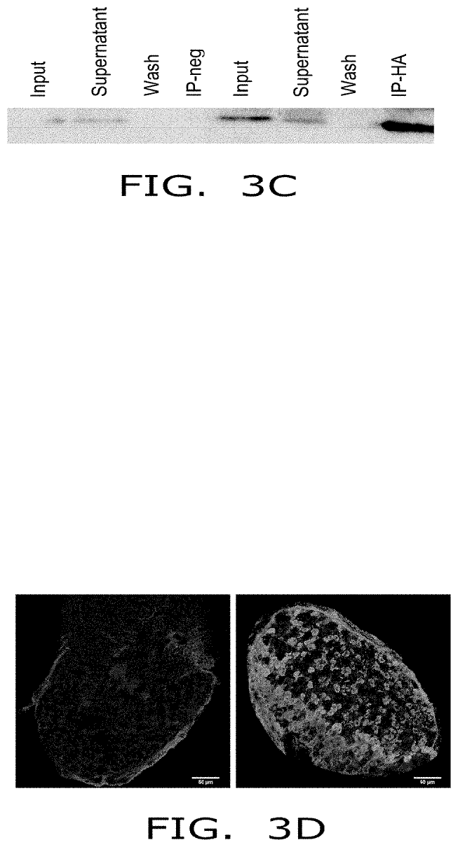

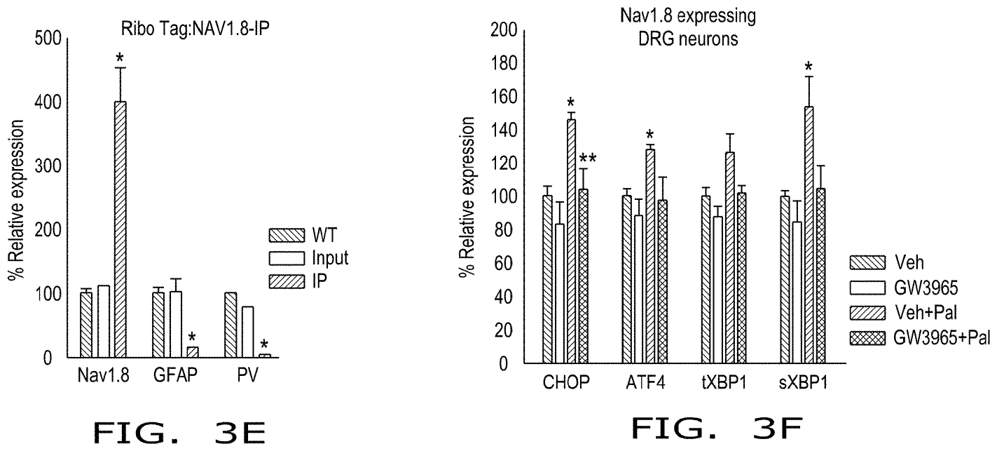

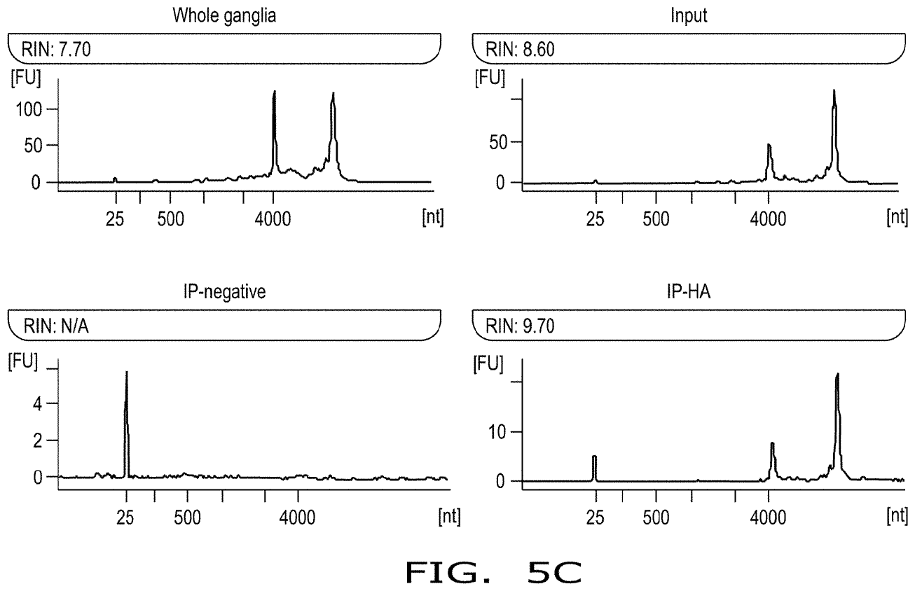

Nav1.8 is a tetrodotoxin-resistant sodium channel expressed exclusively in primary sensory neurons with particularly high levels of expression in nociceptive neurons with small- and medium-sized soma diameters, and are involved in neuropathic pain. Interestingly, the neurons expressing Nav1.8 had been reported as important targets in painful type 2 diabetic neuropathy models. To further evaluate the effect of saturated fatty acids and LXRs on sensory neurons of the DRG, a sensory neuron specific deletion of LXRs (LXRα and LXRβ) (LXRα^{fl/fl}β^{fl/fl}:Nav1.8Cre+/−; LXRabnav) was generated by crossing LXRα^{fl/fl}β^{fl/fl} (LXRab) mice with Nav1.8Cre+/− mice (FIG. 5B). Mice expressing a HA-tagged ribosomal protein (RPL22-HA) specifically in the sensory neurons (RiboTag+/+:Nav1.8Cre+/−) was also generated by crossing RiboTag mice with hemizygous Nav1.8-Cre mice (RiboTag mice procedure, FIG. 3B).

LXRab and LXRabnav mice were fed either WD or NC and assessed for the onset and progression of mechanical allodynia. While both LXRab and LXRabnav mice weighed significantly more than control mice when fed WD (FIG. 5A), WD-fed LXRabnav mice gained significantly less weight than their LXRab counterparts (FIG. 5A). Loss of LXRα and LXRβ in sensory neurons of the DRG further augmented WD-induced allodynia (FIG. 3A), indicating LXRs in the sensory neurons of the DRG regulate WD-induced mechanical allodynia.

To investigate the cell-specific molecular mechanisms underlying this effect, ex-vivo DRG organotypic cultures of WT and RiboTag+/+:Nav1.8Cre+/− were treated with palmitate and GW3965. Sensory neuron specific mRNAs were isolated from DRG of RiboTag+/+:Nav1.8Cre+/−. Bioanalyzer traces (FIG. 5C) showed the purity and integrity of mRNA isolated from immunoprecipitation (IP) of polysomes from RiboTag+/+:Nav1.8Cre+/−.

These investigations verified the presence of HA in the IP sample versus controls (FIGS. 2A-2D and 3C) the presence of HA staining in Nav1.8 expressing neurons of the DRG (FIG. 3D). The expression of a positive control gene (Scn10a/Nav1.8; 3 fold enrichment) and negative control genes (Glial fibrillary acidic protein (GFAP), Parvalbumin (PV)) was further evaluated (FIG. 3E). These data confirm that mRNA from Nav1.8 positive neurons of the DRG was significantly enriched.

The mRNA levels of ER stress markers undergoing translation were analyzed in Nav1.8 expressing neurons. CHOP, ATF4, and sXBP1 mRNA levels in sensory neurons treated with palmitate were increased compared to vehicle controls (FIG. 3F). These increases were blunted by treating with GW3965 (FIG. 3F) suggesting that LXRs regulate lipid-induced ER stress in the sensory neurons of the DRG.

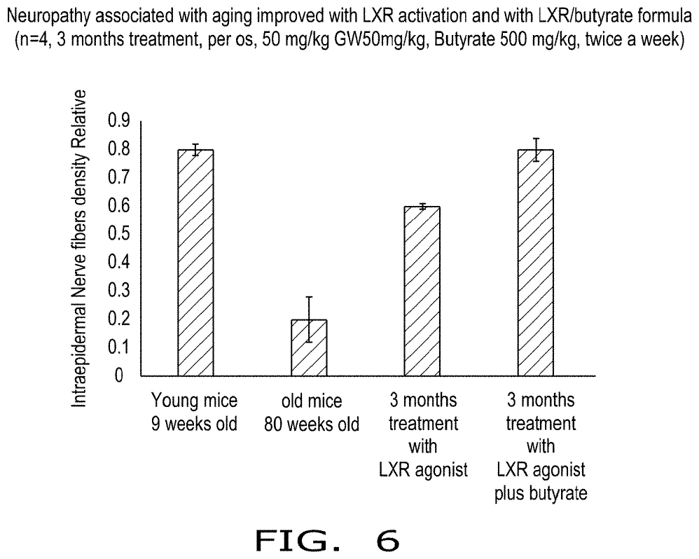

In an additional investigation, 80-week old mice presenting signs of neuropathy (loss of fibers in the skin) were gavaged twice a week during a three-month period with a GW3965 solution or a mixed solution of GW3965 and butyrate using the indicated concentrations reported in FIG. 6. After three months, the mice were euthanized and the intra epidermal nerve fibers density was counted in the skin of their paws. It was observed that the treatment with both solutions increased the number the nerves, but the effect was significantly greater with the mixed solution of GW3965 and butyrate, suggesting that LXR and butyrate signaling triggers an enhanced regeneration mechanism that improves age-induced neuropathy.

Table 1 includes a list of qPCR primers used in investigations described herein.

TABLE 1

| | Forward (5′-3′) | Reverse (5′-3′) |
|---|---|---|
| 18s | AGGACCGCGGTTCTATTTTGT | ATGCTTTCGCTCTGGTCCGTC |
| | TGG | TTG |
| CHOP | CCACCACACCTGAAAGCAGAA | AGGTGAAAGGCAGGGACTCA |
| XBP1 | TGGCCGGGTCTGCTGAGTCCG | GTCCATGGGAAGATGTTCTGG |
| sXBP1 | CTGAGTCCGAATCAGGTGCAG | GTCCATGGGAAGATGTTCTGG |
| usXBP1 | CAGCACTCAGACTATGTGCA | GTCCATGGGAAGATGTTCTGG |
| ATF4 | GGGTTCTGTCTTCCACTCCA | AAGCAGCAGAGTCAGGCTTTC |
| LPCAT3 | TCTGGGGCAAATTTGTGCTG | AGCCACACTTTCATGTTGGC |
| Nav1.8 | TGCTGCAAAGTGAACACCAG | ATGCGGTAACAGGTTTTGCG |
| GFAP | TCAATGCTGGCTTCAAGGAG | AGCGCCTTGTTTTGCTGTTC |
| PPARy1 | GCGGCTGAGAAATCACGTT | TCAGTGGTTCACCGCTTCTT |
| PPARy2 | CACCAGTGTGAATTACAGCA | ACAGGAGAATCTCCCAGAGTT |
| | AATC | C |
| Abca1 | GGTTTGGAGATGGTTATACA | CCCGGAAACGCAAGTCC |
| | ATAGTTGT | |
| PV | GACACCACCTGTAGGGAGGA | AGTACCAAGCAGGCAGGAGA |
| Actin | ACCTTCTACAATGAGCTGCG | CTGGATGGCTACGTACATGG |
| LXRa | AGAGCTTCGTCCACAAAAGC | AGCACGTTGTAATGGAAGCC |
| LXRb | TTGTGGACTTTGCCAAGCAG | TGCATTCTGTCTCGTGGTTG |

In summary, the above-noted investigations used LXR agonist and cell-specific rodent models to provide insights into the cellular and molecular pathogenesis of obesity-associated allodynia and link LXRs with ER stress in DRG neurons. In particular, it was determined that the nuclear receptors LXRs are transcriptionally active in the dorsal root ganglia, are involved in WD-induced allodynia, and locally regulate saturated lipid-mediated ER stress. In addition, it has been shown that LXR agonist treatment delays a western diet-induced allodynia. Studies using the above-described genetically modified models may be used to identify pathways to treat obesity-induced neuropathy and advance our knowledge in the cell-specific function of the LXRs.

Based on the investigations reported above, and because the pain phenotype that was tested mimics the pain observed in obese humans, it was concluded that a pharmaceutical containing an LXR agonist (particularly but not limited to GW3965, T0901317, desmosterol, N,N-dimethyl-3β-hydroxycholenamide (DMHCA) and methylpiperidinyl-3β-hydroxycholenamide (MePipHCA), cholesterol, and hydroxycholesterol) can be administered (e.g., intraperitoneally, intravenous, orally, or topically) to a human with a condition such as metabolic syndrome, obesity, aging, and skin condition (e.g., inflammation of the skin leading to pain that uses the same fibers as allodynia) to successfully treat and improve neuropathy and pain associated therewith. Such a treatment is also believed to be therapeutic for other neuropathies in subjects, for example, fibromyalgia, which involves the same neurons and pain mechanisms as the above-noted conditions. Such benefits can be enhanced if the LXR agonist is used in combination with a form of butyrate, as nonlimiting examples, sodium butyrate, tributyrin, and fibers that increase butyrate production by gut microbiome.

The dose of such a pharmaceutical administered to a subject, particularly a human, in the context of the present invention, should be sufficient to effect a therapeutic response in the subject over a reasonable time frame. Those of ordinary skill in the art will recognize that dosage will depend upon a variety of factors including the condition of the subject, the body weight of the subject, the nature and extent of the subject's symptoms, the kind of concurrent treatment, the frequency of treatment, etc. The size of the dose also will be determined by the route, timing, and frequency of administration as well as the existence, nature, and extent of any adverse side effects that might accompany the administration of the pharmaceutical and the desired physiological effect. Appropriate dosing may be determined empirically from clinical trials, starting with doses that have established safety profiles when used for other applications.

While the invention has been described in terms of specific or particular embodiments and investigations, it should be understood that the invention is not necessarily limited to any embodiment described herein or illustrated in the drawings. It should also be understood that the phraseology and terminology employed above are for the purpose of describing the disclosed embodiments and investigations, and do not necessarily serve as limitations to the scope of the invention. Therefore, the scope of the invention is to be limited only by the following claims.

1. A method of treating neuropathy in a subject, the method comprising administering to the subject a composition comprising a liver X receptor agonist.

2. The method of claim 1, wherein the composition administered includes an amount of a liver X receptor agonist sufficient to control and optionally delay the onset and progression of neuropathy in the subject.

3. The method of claim 1, wherein the composition administered includes an amount of a liver X receptor agonist sufficient to control and optionally delay the onset and progression of diet-induced allodynia in the subject.

4. The method of claim 1, wherein the composition includes a butyrate.

5. The method of claim 1, wherein the liver X receptor agonist is chosen from the group consisting of GW3965, T0901317, desmosterol, N,N-dimethyl-3β-hydroxycholenamide (DMHCA) and methylpiperidinyl-3β-hydroxycholenamide (MePipHCA), cholesterol, and hydroxycholesterol.

6. The method of claim 1, wherein the neuropathy is associated with metabolic syndrome in the subject.

7. The method of claim 6, wherein the composition administered includes an amount of a liver X receptor agonist sufficient to control and optionally delay the onset and progression of neuropathy in the subject.

8. The method of claim 6, wherein the composition administered includes an amount of a liver X receptor agonist sufficient to control and optionally delay the onset and progression of diet-induced allodynia in the subject.

9. The method of claim 6, wherein the composition includes a butyrate.

10. The method of claim 6, wherein the liver X receptor agonist is chosen from the group consisting of GW3965, T0901317, desmosterol, N,N-dimethyl-3β-hydroxycholenamide (DMHCA) and methylpiperidinyl-3β-hydroxycholenamide (MePipHCA), cholesterol, and hydroxycholesterol.

11. A method of treating neuropathy in a subject, the method comprising administering to the subject a composition comprising a liver X receptor agonist in an amount sufficient to control endoplasmic reticulum stress due to the accumulation of unfolded proteins.

12. The method of claim 11, wherein the composition administered includes an amount of a liver X receptor agonist sufficient to control and optionally delay the onset and progression of neuropathy in the subject.

13. The method of claim 11, wherein the composition administered includes an amount of a liver X receptor agonist sufficient to control and optionally delay the onset and progression of diet-induced allodynia in the subject.

14. The method of claim 11, wherein the composition includes a butyrate.

15. The method of claim 11, wherein the liver X receptor agonist is chosen from the group consisting of GW3965, T0901317, desmosterol, N,N-dimethyl-3β-hydroxycholenamide (DMHCA) and methylpiperidinyl-3β-hydroxycholenamide (MePipHCA), cholesterol, and hydroxycholesterol.

16. The method of claim 11, wherein the neuropathy is associated with metabolic syndrome in the subject.

17. The method of claim 16, wherein the composition administered includes an amount of a liver X receptor agonist sufficient to control and optionally delay the onset and progression of neuropathy in the subject.

18. The method of claim 16, wherein the composition administered includes an amount of a liver X receptor agonist sufficient to control and optionally delay the onset and progression of diet-induced allodynia in the subject.

19. The method of claim 16, wherein the composition includes a butyrate.

20. The method of claim 16, wherein the liver X receptor agonist is chosen from the group consisting of GW3965, T0901317, desmosterol, N,N-dimethyl-3β-hydroxycholenamide (DMHCA) and methylpiperidinyl-3β-hydroxycholenamide (MePipHCA), cholesterol, and hydroxycholesterol.
